(19) United States
(12) Patent Application Publication
(10) Pub. No.: US 2005/0037480 A1
(43) Pub. Date: Feb. 17, 2005

(54) Method for culturing organic blue-green algae

(75) Inventor: Chuang Chun Chiueh, Taipei (TW)

Correspondence Address: BACON & THOMAS, PLLC, 625 SLATERS LANE, FOURTH FLOOR, ALEXANDRIA, VA, 22314, US

(73) Assignee: Far East Microalgae Ind. Co., Ltd., Taipei City (TW)

(21) Appl. No.: 10/800,623

(22) Filed: Mar. 16, 2004

(30) Foreign Application Priority Data
Aug. 14, 2003 (TW) 92122412

(51) Int. Cl. C12N 1/20

(52) U.S. Cl. 435/252.1

(57) ABSTRACT

The present invention relates to a method for culturing blue-green algae, comprising the steps of obtaining desired algae species, inoculating the culture medium and carrying out mass culture, and characterized in which said culture medium contains fermented and aerated high-nitrogen organic substance and has a pH of 8 or greater, and is free of inorganic salts, such as carbonate or hydrogen carbonate.

### BACKGROUND OF THE INVENTION

1. Field of the Invention

The present invention discloses a method for culturing organic blue-green algae and the preparation of its culture medium, characterized in which no hydrogen carbonate, carbonate or other pH adjuster or buffer are added in the culture process, and instead, pH level of culture medium suitable for algae growth is achieved using fermented organic matter, agitation and aeration.

2. Description of Related Art

Blue-green algae are single-celled organisms that live in warm, alkaline fresh waters. It is bluish green in color due to the presence of chlorophyll and phycocyanin, and because of its spiral shape, it is also called spirulina. Blue-green algae is rich in nutrients, including gamma-linolenic acid (GLA), linoleic acid, arachidonic acid, vitamin B12, iron, protein, RNA, DNA, chlorophyll, and a blue pigment (phycocyanin) only seen in blue-green algae. Phycocyanin is found to increase the survival rate of rats with liver cancer in experimental studies. Taking blue-green algae in the form of food product helps boost immune system, lower cholesterol, enhance mineral absorption, and cleanse the body of toxins. Blue-green algae is alkaline, which can help regulate the acidic constitution caused by undesirable diet habit, and thereby, prevent and decrease the incidence of chronic diseases.

Dietary algae are typically grown in artificial environment. In light that algae are highly sensitive organisms, the culture technology and process have significant effect on the quality of resulting algae. In the known culture processes for blue-green algae, chemical fertilizers are added to replenish carbon, nitrogen, phosphorus, potassium and other plant nutrients, and inorganic salts, such as carbonate and hydrogen carbonate are also added. In the case of Patent No. CN1218831 which discloses a regulating and controlling method for carbon source and pH value in cultivating spirulina, NaHCO₃ or carbon dioxide is added to the culture medium for precise control of pH so as to promote the growth and propagation of blue-green algae. Another patent No. CN1254012 relates to a method for house cultivating edible fresh spirulina, in which inorganic compounds containing NaHCO₃ are selected as culture medium, which has a pH of 8˜11 after dissolution in water, and desired algae species placed in the culture medium are cultivated under water temperature of 25˜40° C. in an alkaline-resistant and non-toxic installation that provides lighting, heat and agitation. Patent No. RO117388 discloses a mutant of spirulina platensis (Nordst) Geitl CCTE-97/3, culture medium, process and installation for continuous flow cultivation, in which the culture medium contains NaHCO₃, NaNO₃, HCO₃⁻ and other inorganic nutrients. Patent No. JP1037281 provides a culture method for marine blue-green algae, which comprises the steps of adding condensed phosphate with specific concentration to the culture solution, dissociating sufficient iron ion, and maintaining a dissolution state to promote the growth of blue-green algae.

As described above, Na₂CO₃, NaHCO₃, NaH₂PO₄ and other inorganic salts are added as nutrient, pH adjuster or buffer to obtain the desired pH level suitable for algae growth. But those inorganic compounds will be absorbed by algae and the residues of chemical fertilizer in the dietary algae are burdens to human body. It will be environmentally friendly, safer and healthier for human body if dietary algae are grown in an organic environment having the desired pH level without the addition of artificial chemicals. The resulting algae will have higher nutritional value and provide more benefits.

### SUMMARY OF THE INVENTION

To address the drawback of known culture methods for blue-green algae, the important task is to develop a method for culturing pure organic algae that does not require the addition of inorganic salts in the process.

The present invention provides a method of culturing organic blue-green algae.

The present invention further provides a process for preparing culture medium for organic blue-green algae.

Another objective of the present invention is to provide an organic blue-green algae cultured using the aforesaid culture medium.

Yet another objective of the present invention is to provide a culture medium for organic blue-green algae prepared according to the process mentioned above.

Yet another objective of the present invention is to provide a culture medium for organic blue-green algae according to the process for preparing culture medium mentioned above.

The method of culturing organic blue-green algae herein comprises the steps of: obtaining desired algae species; inoculating the culture medium; and carrying out mass culture; characterized in that the culture medium used contains fermented and aerated high-nitrogen organic substance and has a pH of 8 or greater.

The aforesaid high-nitrogen organic substance is preferably high-protein organic matter.

The aforesaid culture medium is free of inorganic salts, such as carbonate or hydrogen carbonate.

The aforesaid culture medium may be further added with edible microorganisms, such as fermenting strains for dairy products (e.g. acidophilus and yeast).

The process for preparing culture medium for organic blue-green algae herein comprises the steps of obtaining high-nitrogen organic substance; carrying out agitation and fermentation; and carrying out aeration and agitation until the pH of culture medium becomes 8 or greater.

The aforesaid high-nitrogen organic substance is preferably high-protein organic matter.

The aforesaid culture medium is free of inorganic salts, such as carbonate or hydrogen carbonate.

The raw material for aforesaid culture medium may be further added with edible microorganisms, such as fermenting strains for dairy products.

According to the method for culturing organic blue-green algae herein, the culture medium having a pH level of ≧8.0 may be achieved by means of aeration and agitation over a specific period of time without the addition of Na₂CO₃, NaHCO₃, NaH₂PO₄, or other inorganic salts as pH adjuster or buffer, so that the entire culture process is carried out in an organic environment and stays free of the contamination of inorganic additives. The resulting organic blue-green algae offer the consumers an organic, safe and healthy choice that may be ingested directly or further processed.

These and other aspects and advantages will become apparent when the DESCRIPTION below is read in conjunction with the accompanying Examples.

### BRIEF DESCRIPTION OF THE DRAWINGS

FIG. 1 shows the flow chart of the method for culturing organic blue-green algae according to the present invention.

FIG. 2 shows the process for preparing culture medium for organic blue-green algae according to the present invention.

FIG. 3 shows the process for preparing culture medium (I) for organic blue-green algae according to the present invention.

FIG. 4 shows the process preparing culture medium (II) for organic blue-green algae according to the present invention.

FIG. 5 shows the process preparing culture medium (III) for organic blue-green algae according to the present invention.

FIG. 6 shows the culture method for organic blue-green algae in Example 4 herein.

### DETAILED DESCRIPTION OF THE INVENTION

It must be noted that as used herein and in the appended claims, the singular forms “a”, “an”, and “the” include plural reference unless the context clearly dictates otherwise. Thus, for example, reference to “an organic blue-green algae” includes a plurality of such organic blue-green algaes, and equivalents thereof known to those skilled in the art, and so forth.

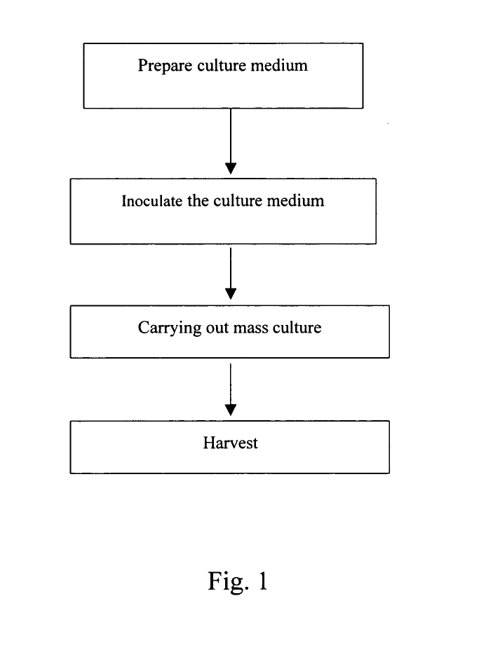

The present invention provides a method for culturing organic blue-green algae as shown in FIG. 1 which comprises the steps of preparing culture medium; inoculating desired algae species to be cultured on the aforesaid culture medium; carrying out mass culture by continuous agitation with pH of culture medium maintained under certain level; and harvesting the resulting biomass of algae by proper means, such as spray drying.

The present invention features the process for preparing the culture medium. Below are descriptions of the preparation method followed by the illustration of examples.

Process for the Preparation of Culture Medium for Organic Blue-Green Algae

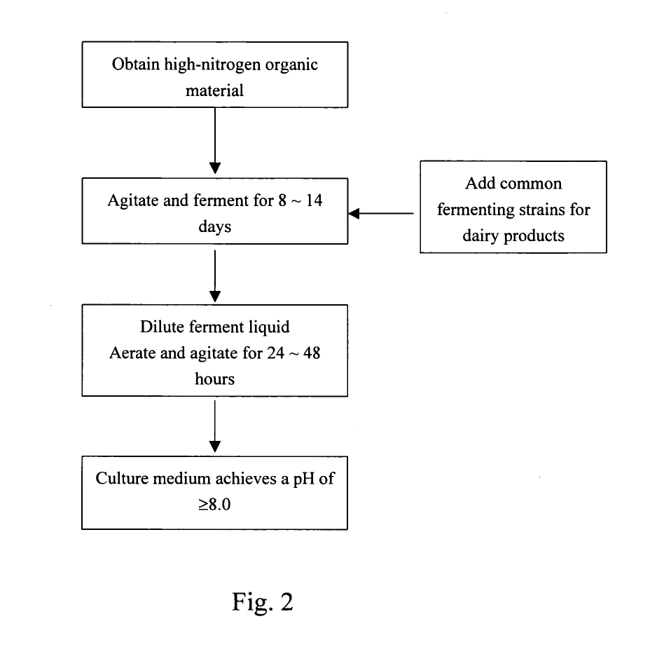

The process for preparing the culture medium for organic blue-green algae herein as depicted in FIG. 2 comprises of the following steps: agitate and ferment high-nitrogen organic material for 8˜14 days, during which fermenting strains for dairy products, such as lactobacillus rhamnosum LGG, lactobacillus acidophilus, streptococcus lactis, bacillus subtilis, brewers yeast or rhodopseudomonas palustris, may be added selectively. After fermentation, dilute the ferment liquid and then subject it to aeration and agitation for 24˜48 hours to obtain a medium with pH ≧8.0 without the need to add inorganic salts, such as Na₂CO₃, NaHCO₃, NaH₂PO₄ as pH adjuster, buffer or nutrients so as to obtain a true organic culture environment.

The following examples are presented in order to more fully illustrate the preferred embodiments of the invention. They should in no way be construed, however, as limiting the broad scope of the invention. While the invention is described and illustrated herein by references to various specific material, procedures and examples, it is understood that the invention is not restricted to the particular material combinations of material, and procedures selected for that purpose. Numerous variations of such details can be implied as will be appreciated by those skilled in the art.

### EXAMPLE 1

### Preparation of Organic Blue-Green Algae Culture Medium (I)

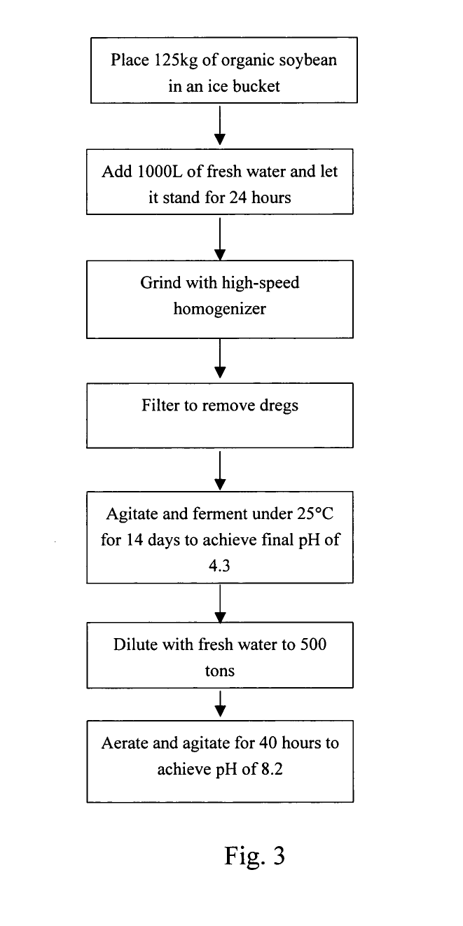

Here a method of preparing culture medium where no fermenting strains are added in the process is described which consists of the steps as depicted in FIG. 3: place 125 kg of organic soybean (from Fu-Yueholi International, Lot No. 040502) in an ice bucket and add 1000 liters of fresh water; after letting it stand for 24 hours, grind the organic soybean using high-speed homogenizer and then filter to remove dregs; agitate and ferment the ground soybean continuously for 14 days under 25° C. to achieve a final pH of 4.3; dilute the ferment liquid with fresh water to 500 tons and then subject it to aeration and agitation for 40 hours until the solution achieves a pH of 8.2.

### EXAMPLE 2

### Preparation of Organic Blue-Green Algae Culture Medium (II)

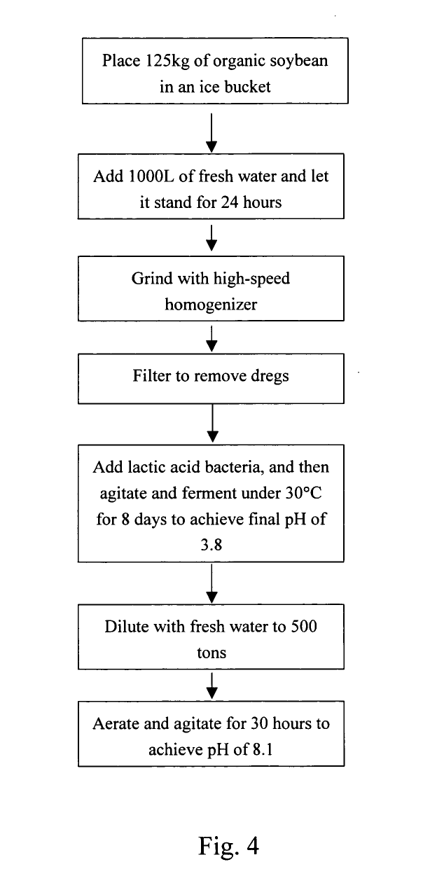

This example provides a method of preparing culture medium where fermenting strains are added in the process which consists of the steps as depicted in FIG. 4: place 125 kg of organic soybean (from Fu-Yueholi International, Lot No. 040502) in an ice bucket and add 1000 liters of fresh water; after letting it stand for 24 hours, grind the organic soybean using high-speed homogenizer and then filter to remove dregs; add lactic acid bacteria (from Chuan Ya Co., Lot No. XAB-35), and then agitate and ferment the ground soybean continuously for 8 days under 30° C. to achieve a final pH of 3.8; dilute the ferment liquid with fresh water to 500 tons and then subject it to aeration and agitation for 30 hours until the solution achieves a pH of 8.1.

### EXAMPLE 3

### Preparation of Organic Blue-Green Algae Culture Medium (III)

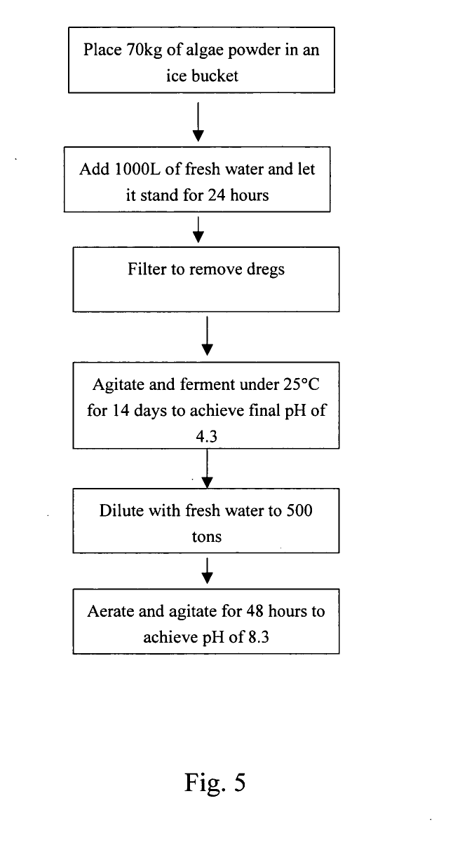

As shown in FIG. 5, place 70 kg of algae powder (obtained by spray drying the harvested algae) in an ice bucket and add 1000 liters of fresh water; after agitation for 24 hours, homogenize the algae solution and filter to remove dregs; agitate and ferment the solution continuously for 14 days under 25° C. to achieve a final pH of 4.3; dilute the ferment liquid with fresh water to 500 tons and then subject it to aeration and agitation for 48 hours until the solution achieves a pH of 8.3.

The three examples below describe the culture of organic blue-green algae using the culture medium obtained in Examples 1, 2 and 3 respectively.

### EXAMPLE 4

### Culture of Organic Blue-Green Algae (I)

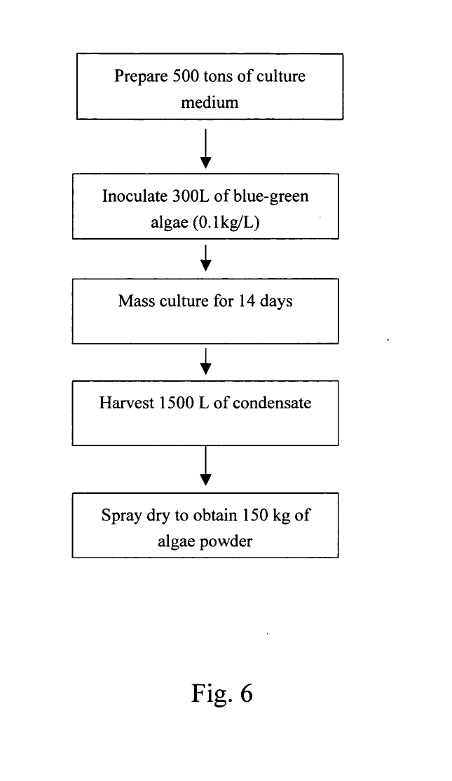

This example provides a method for culturing organic blue-green algae. As shown in FIG. 6, prepare 500 tons of culture medium (I) according to Example 1; inoculate said medium with 300 liters of blue-green algae (density of 0.1 kg per liter); after mass culturing for 14 days, collect the condensate and carry out spray drying to obtain 150 kg of algae powder.

### EXAMPLE 5

### Culture of Organic Blue-Green Algae (II)

This example provides a method for culturing organic blue-green algae which is the same as that described in Example 4: prepare 500 tons of culture medium (II) according to Example 2; inoculate said medium with 300 liters of blue-green algae (density of 0.1 kg per liter); after mass culturing for 14 days, collect the condensate and carry out spray drying to obtain certain quantity of algae powder.

### EXAMPLE 6

### Culture of Organic Blue-Green Algae (III)

This example provides a method for culturing organic blue-green algae which is the same as that described in Example 4: prepare 500 tons of culture medium (III) according to Example 3; inoculate said medium with 300 liters of blue-green algae (density of 0.1 kg per liter); after mass culturing for 14 days, collect the condensate and carry out spray drying to obtain certain quantity of algae powder.

A comparison of Examples 4˜6 is illustrated in the table below.

| Example | Example 4 | Example 5 | Example 6 |
|---|---|---|---|
| Culture medium | Culture | Culture | Culture |
| | medium (I) | medium (II) | medium (III) |
| Yield (blue-green algae) | 0.29 g/L | 0.32 g/L | 0.35 g/L |

The preferred embodiments of the present invention as disclosed above are not meant to limit this invention. All modifications and alterations made by those familiar with the skill without departing from the spirits of the invention and appended claims shall remain within the protected scope and claims of the invention.

1. A method for culturing organic blue-green algae, comprising the steps of:
obtaining desired algae species;
inoculating the culture medium; and
carrying out mass culture;
characterized in that said culture medium contains high-nitrogen organic substance, which is fermented and aerated to give the culture medium a pH of 8 or greater.

2. The method according to claim 1, wherein said culture medium is not added with inorganic salts.

3. The method according to claim 2, wherein said culture medium is not added with carbonate or hydrogen carbonate.

4. The method according to claim 1, wherein said culture medium may be further added with edible microorganisms.

5. The method according to claim 1, wherein said high-nitrogen organic substance is high-protein organic matter.

6. A process for preparing culture medium for organic blue-green algae, comprising the steps of:
obtaining high-nitrogen organic substance;
carrying out agitation and fermentation; and
carrying out aeration and agitation until the medium achieves a pH of 8 or greater.

7. The process according to claim 6, wherein said high-nitrogen organic substance is high-protein organic matter.

8. The process according to claim 6, wherein said culture medium is not added with inorganic salts.

9. The process according to claim 8, wherein said culture medium is not added with carbonate or hydrogen carbonate.

10. The process according to claim 6, wherein said culture medium may be further added with edible microorganisms.

11. An organic blue-green algae cultured according to the method of claim 1.

12. A culture medium for organic blue-green algae prepared according to the process of claim 6.

13. An organic blue-green algae cultured in the culture medium according to claim 12.
